(19) United States
(12) Patent Application Publication
(10) Pub. No.: US 2008/0306462 A1
(43) Pub. Date: Dec. 11, 2008

(54) DISPOSABLE MULTILAYERED LOW FRICTION DOUBLE LUBRICATED BED PAD

(76) Inventors: Arnold Bruckner, Brooklyn, NY (US); Holly M. Lidowski, West Babylon, NY (US); Leslie Strauss, Dix Hill, NY (US); Karl K. Eilender, New York City, NY (US)

Correspondence Address: LAW OFFICE OF MARC D. MACHTINGER, LTD., 750 W. LAKE COOK ROAD, SUITE 350, BUFFALO GROVE, IL, 60089, US

(21) Appl. No.: 11/759,492

(22) Filed: Jun. 7, 2007

(51) Int. Cl. A61F 13/00

(52) U.S. Cl. 604/365

(57) ABSTRACT

A disposable multilayered double lubricated bed pad is disclosed. Specifically, a lubricated pad for preventing and treating bedsores on a person's body and for managing discharged body fluids is disclosed. Among other things, the disclosed pad has absorbing layers and means for managing discharged body fluids away from a person's body to the absorbing layers. The disclosed pad also has a lubricating layer and is constructed to minimize friction between a person's body and a supporting surface. The disclosed pad is also treated with a lubricating, skin protecting, and moisturizing emollient for the treatment and prevention of pressure sores The emollient can be microencapsulated in the lubricating layer.

### FIELD OF THE INVENTION

The present invention relates to low friction bed pads. More specifically, the present invention relates to an improved disposable multilayer bed pad for reducing or preventing skin breakdown, bedsores, pressure sores, decubitus ulcers and similar lesions caused by pressure, friction, and shearing forces as well as moisture applied to a person's body. Even more specifically, the present invention relates to an improved disposable multilayer bed pad having at least two lubricants. Furthermore, the present invention relates to a bed pad for managing fluids discharged from the body of a person, such as urine from a bedridden or wheelchair bound patient.

### BACKGROUND OF THE INVENTION

It is well established that recent advances in medicine have resulted in longer life expectancies. These changes in life expectancy when coupled with changes in population levels have resulted in a population of elderly people that is much greater than ever before. In the United States, it is an accepted act that these longer life expectancies carry with them an ever increasing cost of health care. Prevention of untoward medical conditions, such as skin breakdown, can save million of heath care dollars annually.

Skin breakdown, commonly known as bedsores, pressure sores, or decubitus ulcers (hereinafter collectively referred to as bedsores), are ancient problems which recently have begun to reach catastrophic proportions due to the growing population of elderly people. Bedsores are open ulcerations which generally appear in the skin which covers a bony prominence, such as the hip, coccyx, heel, elbow, and head. Ulceration can occur because of compression of tissue between external sources of pressure, friction, and shearing against bony prominences beneath the skin.

The vast majority of bedsores can be prevented, yet in the United States conservative estimates indicate the well over two million people in hospitals and nursing care facilities suffer from these lesions. Due to the ever increasing number of patients at risk for, and suffering from, bedsores, and the increased costs associated with the treatment of these sores, the need to prevent bedsores is becoming more acute.

The main causes of bedsores are the forces of pressure, friction, and shearing. Moisture, particularly from bodily fluids, is also an important factor in the formation of bedsores. Other factors, such as whether a patient is malnourished, incontinent, and even mentally impaired can contribute to the rapid development of bedsores. For skin that is delicate, dry, or compromised by any acute or chronic illness, as is common in elderly patients, these factors work together toward the rapid formation of bedsores. For example, the skin of aged people tends to be increasingly sensitive and tends to be less supple, less hydrated, and even more erythematous (abnormally red due to inflammation and capillary congestion) than that of younger people. Studies indicate that bedsores can start to develop in as short a period of time as between one to six hours.

Friction is the resistance to sliding motion of two bodies pressed against one another. The general term friction encompasses static friction and dynamic friction. Static friction results from the resistance to motion in overcoming inertia. Dynamic friction is created by the irregularities of the two surfaces interlocked with one another. A significant force is required to overcome status friction and thus to obtain sliding movement of two bodies with respect to one another. Static friction ceases to be a significant factor after sliding motion has been achieved between the two bodies. For instance, when a patient is slipping down against sheets while the patient is sitting up in bed, static friction occurs between the skin and the sheets. As another example, consider a patent being positioned in a bed by sliding across the bed linens. In this example, the patient's skin is subject to dynamic friction between the skin and the linens.

Shearing forces are the internal forces within the skin and supporting tissues that result from the subtle and not so subtle movements of the patient while the patient remains stationary with respect to the bed or wheelchair. For example, when a bedridden patient moves his or her arms or twists his or her shoulders, but their hips remain stationary, specifically, the skin on the patient's buttocks remains stationary on the bed, shear forces are created in the skin tissue and supporting tissues. As a result of these shearing forces, capillaries become damaged, distorted, crimped and occluded. This leads to ischemia (deprivation of blood supply to an area) of tissue, which can lead to an area of dead skin, which can develop into a bedsore. Additionally, when friction and shearing occur there is also a build up of heat, which is caused by the rubbing irritation of the skin's surface. The increased heat can lead to microscopic openings in the skin surface, which themselves are susceptible to infection or other irritants.

Furthermore, the introduction of liquid, such as from an incontinent patient, can accelerate bedsore formation. The presence of liquid softens the skin and leads to the separation of the layers of the skin. These factors contribute to the formation of bedsores.

In U.S. Pat. No. 4,572,174, which issued Feb. 25, 1986, we disclosed a bed pad structure intended to relieve the pressure, friction, and shear forces which are now recognized as principal causes of bedsores, pressure sores and decubitus ulcers in bedridden and chair ridden patients. We disclosed a low friction bed pad structure having a pouch portion between a woven fabric upper porous sheet and a lower flexible nonporous sheet. The pouch portion permitted insertion and removal of a lubricated sheet which exuded lubricant that seeped through the pores in the upper sheet to a patient's body when it applied pressure to the pad being held stationary on a bed, to reduce friction between the patient's body and the bed or other supporting surface to which the pad was attached.

Some of the shortcomings of the pads disclosed in U.S. Pat. No. 4,572,174 were addressed in U.S. Pat. No. 4,959,059, which issued Sep. 23, 1990. In U.S. Pat. No. 4,959,059, we disclosed a multilayer low friction ambulatory pad for treating or preventing bedsores and pressure sores and for managing fluids discharged from a person's body. The pad has a first slippery nonporous layer on which is a moisture absorbent second layer. A slippery, thin, porous third layer is on the second layer. The three layers are peripherally bonded to form a continuous seam which permits unbonded areas of the layers to slide slightly with respect to each other. The third layer may be sprayed with a filmy, dry, slippery fourth layer. A fifth layer of lubricating material, which may be a microencapsulated lubricant or a free lubricant, is applied to the third and fourth layers to form a very slippery top surface. The fourth and fifth layers do not clog the pores of the third layer, thus allowing passage of air and fluids to the absorbent second layer. In some variants of the pad, the absorbent layer is omitted. The pad can be discarded after a single use.

Despite the improvements disclosed in U.S. Pat. No. 4,959,059, there remains a need for an improved disposable multilayered bed pad. Specifically, there remains a need for an improved disposable multilayered bed pad for reducing or preventing bedsores caused by pressure and friction applied to a person's body. Furthermore, there remains a need for an improved disposable multilayered bed pad for managing fluids discharged from the body of a person.

### SUMMARY

In view of the deficiencies described above, it is an object of the present invention to provide an improved disposable multilayered bed pad. Specifically, it is an object of the present invention to provide an improved disposable multilayered bed pad for reducing or preventing bedsores caused by pressure and friction applied to a person's body. Even more specifically, it is an object of the present invention to provide an improved disposable multilayered low friction bed pad having at least two lubricants.

It is a further object of the present invention to provide an improved disposable multilayered bed pad for managing fluids discharged from the body of a person.

It is a further object of the present invention to provide an improved disposable multilayered bed pad that is designated for use by those patients who are at risk for developing bedsores caused by any chronic or acute condition that debilitates and may cause a resulting number of variable factors that contribute to the development of bedsores.

It is a further object of the present invention to provide an improved multilayered bed pad that is designed to diminish risk factors that contribute to the development of bedsores. Some of these risk factors include friction forces, shearing forces, and moisture management.

The present invention is a disposable multilayered low friction double lubricated pad for preventing and treating bedsores on a person's body and for managing discharged body fluids. The pad has a bottom first layer which is made from a flexible nonporous film. The pad has an adhesive second layer. The pad has a third layer made of a cellulose tissue carrier. Adhesive second layer bonds the bottom first layer and the third layer together.

The pad has a fourth layer, which has at least two sub-layers. The first sub-layer is made from about 60% porous hydrophilic bleached pulp and about 40% highly absorbent fibrous materials, such as a polymer. The second sub-layer is made from about 40% porous hydrophilic bleached pulp and about 60% highly absorbent fibrous materials, such as a polymer.

The pad has a fifth layer which is wadding of highly absorbent cellulose. The pad has a sixth layer comprising a gram per square meter porous blue polypropylene spun bond acquisition layer, a Guidant indicator and absorbent layer.

An embossing process can be used to connect the bottom first layer, the adhesive second layer, the third layer, the forth layer, the fifth layer, and the sixth layer. This embossing process, preferably done in a pattern, such a diamond pattern, helps manage urine away from the patient and into to the absorbent materials, such as highly absorbent fibrous materials, thus keeping liquids away from the patient's skin.

The pad has a seventh layer made of spun bond fibrous polypropylene material. The pad has a lubricating eighth layer applied to the seventh layer. In various preferred embodiments, this lubricating eighth layer includes silicone. Lubricating eighth layer serves to create an almost friction-free film between the seventh layer and the ninth layer. The pad has a ninth layer which is a lubricating, skin protecting, and moisturizing emollient applied to the lubricating eighth layer.

In various preferred embodiments, the lubricating eighth layer and the ninth layer can be applied to the seventh layer simultaneously using a spray system, such as a swirl or “tornado” inducing spray system, which results in the emollient of the ninth layer being microencapsulated within silicone beads of the lubricating eighth layer.

Marginal areas of the bottom first layer, the third layer, the fourth layer, the fifth layer, the sixth layer, and the seventh layer are nondetachably bonded together, such as by gluing, in a continuous seam all around their joined peripheries. This bonding of the joined peripheries allows limited sliding movement between unbonded areas of the layers. Thus the layers cooperate to minimize sliding friction and shear forces between the person's body and a supporting surface, such as a bed or wheelchair, when the pad placed between the person and the supporting surface.

In various preferred embodiments, the pad can also have a plurality of tape tabs. The tape tabs can be used to secure to the pad to a bed or other supporting surface. Tape tabs can also be used to wrap the pad about a portion of a person's body and secure the pad in place.

Other features and advantages of the invention will be apparent from the following detailed description taken in conjunction with the following figures, wherein like reference numerals represent like features.

### BRIEF DESCRIPTION OF THE DRAWINGS

FIG. 1 is a perspective view of a pad according to the present invention.

FIG. 2 is a perspective view of a pad according to the present invention installed on bed.

FIG. 3 is an exploded view of a pad according to the present invention showing the layers that make up the pad.

FIG. 4 is a sectional side view of a pad according to the present invention showing the layers that make up the pad.

FIG. 5 is a close up view of the fourth layer of a pad according to the present invention showing the sub-layers contained therein.

### DETAILED DESCRIPTION OF THE INVENTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspect of the invention to the embodiments illustrated.

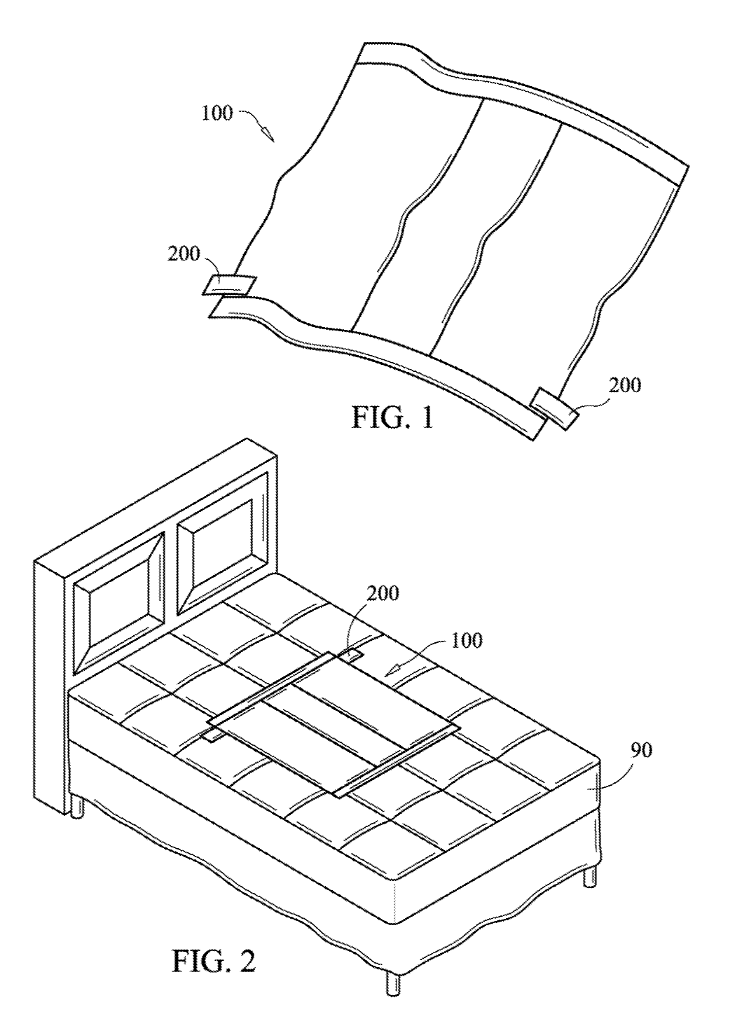

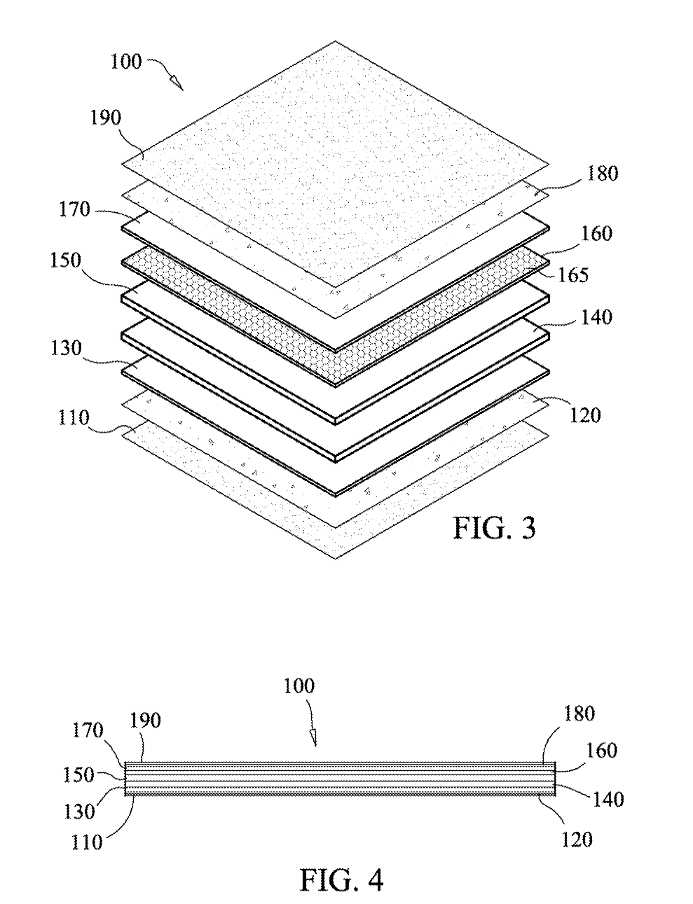

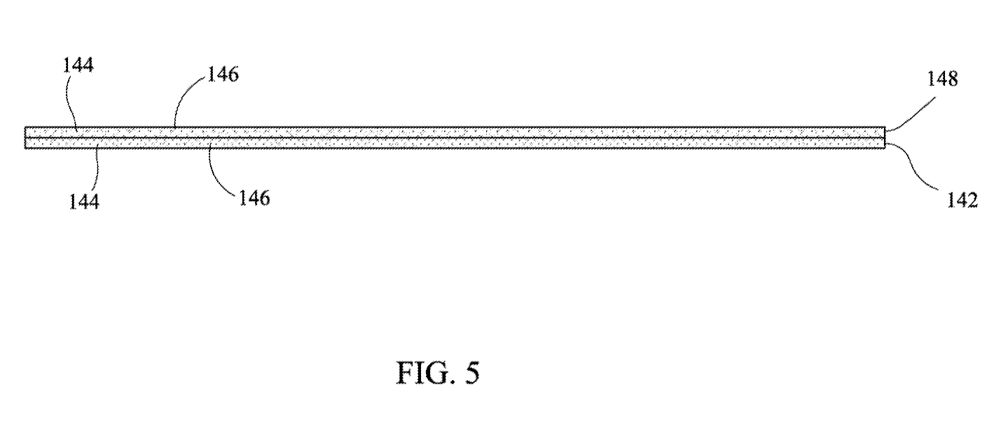

The present invention is a disposable multilayered low friction double lubricated pad for preventing and treating bedsores on a person's body and for managing discharged body fluids. FIG. 1 is a perspective view of a pad according to the present invention. FIG. 2 is a perspective view of a pad according to the present invention installed on a bed. FIG. 3 is an exploded view of a pad according to the present invention showing the layers that make up the pad. FIG. 4 is a sectional side view of a pad according to the present invention showing the layers that make up the pad.

The pad 100 has a bottom first layer 110 which is made from a flexible nonporous film. Bottom first layer 110 can be made from any suitable material. Preferably, bottom first layer 110 is constructed from a thin, flexible, lightweight, strong, nonporous, slippery material, such as a polyethylene film.

The pad 100 has an adhesive second layer 120. Adhesive second layer 120 can be applied to bottom first layer 110 in any suitable manner. In various preferred embodiments, adhesive second layer 120 is sprayed onto bottom first layer 110. The spraying of adhesive second layer 120 can use practices known in the industry, such as spraying adhesive second layer 120 in a pattern, specifically a swirl pattern, on to bottom first layer 110.

The pad 100 has a third layer 130 made of a cellulose tissue carrier. Adhesive second layer 120 bonds the bottom first layer 110 and the third layer 130 together.

The pad 100 has a fourth layer 140 which has at least two sub-layers FIG. 4 is a close up view of the fourth layer of a pad according to the present invention showing the sub-layers contained therein. The first sub-layer 142 is made from about 60% porous lydrophilic bleached pulp 144 and about 40% highly absorbent fibrous materials 146. The second sub-layer 148 is made from about 40% porous hydrophilic bleached pulp 144 and about 60% highly absorbent fibrous materials 146. Porous hydrophilic bleached pulp 144 can be, for example, Bleached Kraft Pulp from Weyerhaeuser Company. The highly absorbent fibrous materials 146 can be, for example, Hysorb 8100 from BASF Corporation or XUS 40690.01 super absorbent polymer from Dow Chemical.

The pad 100 has a fifth layer 150 which is wadding of highly absorbent cellulose.

The pad 100 has a sixth layer 160 comprising a 10 gram per square meter porous blue polypropylene spun bond acquisition layer, a Guidant indicator and absorbent layer. The Guidant indicator and absorbent layer serves to assist in placement of the pad 100 by providing a visual indication of the centerline or other indicating marks of the pad 100.

An embossing process can be used to connect the bottom first layer 110, the adhesive second layer 120, the third layer 130, the forth layer 140, the fifth layer 150, and the sixth layer 160. This embossing process, preferably done in a pattern, such a diamond pattern 165, helps manage urine or other bodily fluids away from the patient and into to the absorbent materials, such as highly absorbent fibrous materials 146, thus keeping liquids away from the patient's skin.

The pad 100 has a seventh layer 170 made of spun bond fibrous polypropylene material. In various preferred embodiments, the seventh layer 170 is about 30 inches wide. Preferably the seventh layer 170 extends past the peripheries of the other layers and can partially wrap around the peripheries of the other layers.

The pad 100 has a lubricating eighth layer 180 applied to the seventh layer. In various preferred embodiments, this lubricating eighth layer 180 includes silicone. For example, lubricating eighth layer can be made from Emulsion 365 from Dow Corning and Akorex L. Lubricating eighth layer 180 serves to create an almost friction-free film between the seventh layer 170 and the ninth layer 190, discussed below. Lubricating eighth layer 180 is applied in an amount effective to reduce friction, but not saturate the seventh layer 170 and thus permit liquid to pass through the seventh layer 170 into the layers below. This effective amount is in the range of about 0.1 grams per square into to about 1.0 grams per square inch.

The pad 100 has a ninth layer 190 comprising a lubricating, skin protecting, and moisturizing emollient applied to the lubricating eighth layer 180. The emollient of the ninth layer is applied in an amount effective to treat and reduce the possibilities of a patient developing bed sores. This effective amount is in the range of about 0.1 grams per square into to about 1.0 grams per square inch.

In various preferred embodiments, the lubricating eighth layer 180 and the ninth layer 190 are applied to the seventh layer 170 simultaneously using a spray system, such as a swirl or “tornado” inducing spray system, which results in the emollient of the ninth layer 190 being microencapsulated within silicone beads of the lubricating eighth layer 180. The results of this microencapsulating process means that the emollient of the ninth layer 190 is not exposed while pad 100 is stored. The weight of a patient being placed on the pad 100 releases the emollient of the ninth layer 190 from the silicone beads of the lubricating eighth layer 180.

Marginal areas of the bottom first layer 110, the third layer 130, the fourth layer 140, the fifth layer 150, the sixth layer 160, and the seventh layer 170 are nondetachably bonded together, such as by gluing, in a continuous seam all around their joined peripheries. This bonding of the joined peripheries allows limited sliding movement between unbonded areas of the layers. Thus the layers cooperate to minimize sliding friction and shear forces between the person's body and a supporting surface, such as a bed 90 or wheelchair, when the pad 100 placed between the person and the supporting surface.

In various preferred embodiments, the pad 100 can also have a plurality of tape tabs 200. The tape tabs 200 can be used to secure to the pad 100 to a bed 90 or other supporting surface. Tape tabs 200 can also be used to wrap the pad 100 about a portion of a person's body and secure the pad 100 in place.

While specific embodiments have been illustrated and described, numerous modifications come to mind without significantly departing from the spirit of the invention and the scope of protection is limited by the scope of the accompanying claims.

What is claimed is:

1. A lubricated pad for preventing and treating bedsores on a person's body and for managing discharged body fluids comprising:
a bottom first layer comprising a flexible nonporous film,
an adhesive second layer,
a third layer comprising a cellulose tissue carrier, wherein said adhesive second layer bonds said bottom first layer and said third layer together,
a fourth layer comprising a first sub-layer and a second sub-layer, said first sub-layer comprising about 60% porous hydrophilic bleached pulp and about 40% highly absorbent fibrous materials, said second sub-layer comprising about 40% porous hydrophilic bleached pulp and about 60% highly absorbent fibrous materials,
a fifth layer comprising wadding of highly absorbent cellulose,
a sixth layer comprising a 10 gram per square meter porous blue polypropylene spun bond acquisition layer, a Guidant indicator and absorbent layer,
wherein said first, second, third, fourth, fifth, and sixth layers undergo a patterned embossing process connecting said sixth layer to said first, second, third, fourth, and fifth layers,
a seventh layer comprising spun bond fibrous polypropylene material,
a lubricating eighth layer applied to said seventh layer,
a ninth layer comprising a lubricating, skin protecting, and moisturizing emollient applied to said lubricating eighth layer, and
means for nondetachably bonding together registering marginal areas of said bottom first layer, said third layer, said fourth layer, said fifth layer, said sixth layer, and said seventh layer in a continuous seam all around their joined peripheries to allow limited sliding movement between unbonded areas of said layers, where said layers cooperate to minimize sliding friction and shear forces between said person's body and a supporting surface with said pad disposed therebetween.

2. The lubricating pad for preventing and treating bedsores on a person's body and for managing discharged body fluids according to claim 1 further comprising a plurality of tape tabs

3. The lubricating pad for preventing and treating bedsores on a person's body and for managing discharged body fluids according to claim 1 wherein said porous hydrophilic bleached pulp of said forth layer comprises Bleached Kraft Pulp.

4. The lubricating pad for preventing and treating bedsores on a person's body and for managing discharged body fluids according to claim 1 wherein said patterned embossing process comprises a diamond patterned embossing process.

5. The lubricating pad for preventing and treating bedsores on a person's body and for managing discharged body fluids according to claim 1 wherein said seventh layer is about thirty (30) inches wide.

6. The lubricating pad for preventing and treating bedsores on a person's body and for managing discharged body fluids according to claim 1 wherein said lubricating eighth layer comprises silicon.

7. The lubricating pad for preventing and treating bedsores on a person's body and for managing discharged body fluids according to claim 1, wherein said means for nondetachably bonding together registering marginal areas of said bottom first layer, said third layer, said fourth layer, said fifth layer, said sixth layer, and said seventh layers comprises glue.

8. The lubricating pad for preventing and treating bedsores on a person's body and for managing discharged body fluids according to claim 1, wherein said lubricating eighth layer comprises Dow Corning 365 NF Emulsion and Akorex L.

9. The lubricating pad for preventing and treating bedsores on a person's body and for managing discharged body fluids according to claim 1, wherein said lubricating eighth layer comprises an effective amount to reduce friction between said seventh layer and said ninth layer.

10. The lubricating pad for preventing and treating bedsores on a person's body and for managing discharged body fluids according to claim 9, wherein said effective amount is in the range of about 0.1 grams per square inch to about 1.0 grams per square inch.

11. The lubricating pad for preventing and treating bedsores on a person's body and for managing discharged body fluids according to claim 1, wherein said ninth layer comprises an effective amount of emollient in the range of about 0.1 grams per square inch to about 1.0 grams per square inch.

12. The lubricating pad for preventing and treating bedsores on a person's body and for managing discharged body fluids according to claim 1, wherein said lubricating eighth layer and said ninth layer are simultaneously sprayed onto said seventh layer, wherein said emollient of said ninth layer becomes microencapsulated in said eighth layer.
